(19) United States
(12) Patent Application Publication
(10) Pub. No.: US 2016/0319443 A1
(43) Pub. Date: Nov. 3, 2016

(54) Sequestration Of Carbon Dioxide By Binding It As Alkali Carbonate

(71) Applicant: SIEMENS AKTIENGESELLSCHAFT, München (DE)

(72) Inventors: Guenter Schmid, Hemhofen (DE); Dan Taroata, Erlangen (DE)

(73) Assignee: Siemens Aktiengesellschaft, Muenchen (DE)

(21) Appl. No.: 15/102,604

(22) PCT Filed: Dec. 3, 2014
(86) PCT No.: PCT/EP2014/076389

§ 371 Date: Jun. 8, 2016

(30) Foreign Application Priority Data
Dec. 10, 2013 (DE) 10 2013 225 419.9

(51) Int. Cl. C25B 1/26; C07C 1/04; B01D 53/81; C07C 29/151; B01D 53/32; B01D 53/62

(52) U.S. Cl. CPC C25B 1/26; B01D 53/326; B01D 53/62; B01D 53/81; C07C 29/1518; C07C 1/0485; B01D 2251/304; B01D 2251/306; B01D 2257/504

(57) ABSTRACT

A method for producing chlorine and for storing carbon dioxide includes electrolytically reacting a compound of the formula M+Cl− being to produce M and Cl2, wherein M+=Na, K or a mixture thereof, reacting the produced M with carbon dioxide and optionally H2O produce to M2CO3 and/or MHCO3, and storing the produced M2CO3 and/or MHCO3.

### CROSS-REFERENCE TO RELATED APPLICATIONS

This application is a U.S. National Stage Application of International Application No. PCT/EP2014/076389 filed Dec. 3, 2014, which designates the United States of America, and claims priority to DE Application No. 10 2013 225 419.9 filed Dec. 10, 2013, the contents of which are hereby incorporated by reference in their entirety.

### TECHNICAL FIELD

The present invention relates to a method for producing chlorine and storing carbon dioxide, wherein a compound of the formula M⁺Cl⁻ is reacted electrolytically to M and Cl₂, wherein M⁺=Na, K or a mixture thereof, M is reacted with carbon dioxide and optionally H₂O to M₂CO₃ and/or optionally to MHCO₃, and M₂CO₃ and/or optionally MHCO₃ is stored, and to a device for carrying out such a method.

### BACKGROUND

At present, approximately 80% of the worldwide energy requirement is covered by burning fossil fuels. In 2011, about 34,032.7 million metric tons of carbon dioxide (CO₂) were emitted into the atmosphere worldwide as a result of these combustion processes. Releasing carbon dioxide in this manner is the simplest way to dispose of even large amounts of CO₂ (lignite-fired power plants over 50,000 t per day).

The CO₂ can additionally be separated off by various methods or used as a raw material.

For example, the carbon dioxide is washed out of flue gas by means of monoethanolamine (MEA). The MEA-CO₂ complex is then cleaved at elevated temperature. Pure CO₂ can thus be stored or permanently disposed of in gas caverns or above-ground tanks, for example. The chemical industry also uses CO₂, for example in order to replace some of the monomer units in polymers by polycarbonate. In the plastics foam so produced, better properties are in some cases observed than without the carbonate additions. There are also known from DE102008031437-A1, WO2010000681-A2, WO2010000681-A3, EP2294643-A2, US20110113844-A1, CN102077395-A methods in which alkali metals, by reaction with CO₂, yield thermal energy suitable for power plants and at the same time valuable intermediates for the chemical industry. However, such methods will not necessarily be able to capture all the carbon dioxide that is produced, and retrofitting in existing plants is in some cases also too expensive.

Further refinements of the method are known from WO2012/038330 and WO2013/156476.

Discussion about the negative effects of the greenhouse gas CO₂ on the climate has led to consideration of the recycling of CO₂. From a thermodynamic point of view, CO₂ is very low and can therefore be reduced again to usable products only with difficulty. Permanent storage of the CO₂ by introducing it into underground gas caverns is therefore considered (carbon capture & storage). The general public has not accepted this form of permanent CO₂ storage because there is the risk that the gas, which has a suffocating effect, will escape and may result in a considerable health risk.

### SUMMARY

One embodiment provides a method for producing chlorine and storing carbon dioxide, wherein (a) a compound of the formula M⁺Cl⁻ is reacted electrolytically to M and Cl₂, wherein M⁺=Na, K or a mixture thereof; (b) M is reacted with carbon dioxide and optionally H₂O to M₂CO₃ and/or optionally to MHCO₃; and (c) M₂CO₃ and/or optionally MHCO₃ is stored.

In one embodiment, the reaction of M with carbon dioxide and optionally H₂O in step (b) is carried out by burning M in an atmosphere comprising carbon dioxide and optionally H₂O.

In one embodiment, the energy for the electrolytic reaction of M⁺Cl⁻ to M and Cl₂ consists substantially of excess energy from renewable energies.

In one embodiment, the compound of the formula M⁺Cl⁻ is obtained from waste products of the chemical industry.

In one embodiment, the electrolysis in step a) is carried out by fused salt electrolysis of a mixture comprising a compound of the formula M⁺Cl⁻.

In one embodiment, in the reaction in step (b) carbon monoxide is also formed, which is optionally reacted to give further chemical products.

In one embodiment, the electrolysis in step (a) is carried out by electrolysis of an aqueous solution of the compound of the formula M⁺Cl⁻ with the formation of hydrogen.

In one embodiment, in the reaction in step (b) carbon monoxide is also produced, which is reacted with the hydrogen from step a) to give further chemical products.

Another embodiment provides a device for producing chlorine and storing carbon dioxide, comprising an electrolysis device, which is designed to react a compound of the formula M⁺Cl⁻ electrolytically to M and Cl₂, wherein M⁺=Na, K or a mixture thereof; a first feed device for M⁺Cl⁻, which is designed to feed M⁺Cl⁻ to the electrolysis device; a first discharge device for Cl₂, which is designed to remove Cl₂ from the electrolysis device; a second discharge device for M, which is designed to remove M from the electrolysis device; a first reactor for reacting carbon dioxide with M, which is designed to react M with carbon dioxide and optionally H₂O to M₂CO₃ and/or optionally to MHCO₃; a second feed device for M, which is connected to the second discharge device for M and is designed to feed M to the first reactor; a third feed device for carbon dioxide and optionally H₂O, which is designed to feed carbon dioxide and optionally H₂O to the first reactor; a third discharge device for M₂CO₃ and/or optionally MHCO₃, which is designed to remove M₂CO₃ and/or optionally MHCO₃ from the first reactor; and a storage device for storing M₂CO₃ and/or optionally MHCO₃, which is designed to store M₂CO₃ and/or optionally MHCO₃ which comes from the third discharge device.

In one embodiment, the device further comprises a device for generating renewable energy, which is designed to supply electrical energy to the electrolysis device.

In one embodiment, the first reactor has a burner for burning M with carbon dioxide and optionally H₂O.

In one embodiment, the electrolysis device is a fused salt electrolysis device.

In one embodiment, the electrolysis device is designed such that an aqueous solution of M⁺Cl⁻ is electrolyzed, further comprising a fourth discharge device for hydrogen, which is designed to remove hydrogen from the electrolysis device.

In one embodiment, the device further comprises a fourth feed device for hydrogen, which is connected to the fourth discharge device for hydrogen and is designed to feed hydrogen to the first reactor.

### BRIEF DESCRIPTION OF THE DRAWINGS

Example aspects and embodiments of the invention are described in detail below with reference to the drawings, in which:

FIG. 1 shows schematically a first exemplary embodiment of the present invention;

FIG. 2 shows schematically a second exemplary embodiment of the present invention; and

FIG. 3 shows schematically a third exemplary embodiment of the present invention.

### DETAILED DESCRIPTION

Embodiments of the invention provide a method of storing carbon dioxide safely and efficiently.

The inventors have found that sequestration of carbon dioxide is possible by using alkali metal salts of Na and/or K while at the same time producing chlorine, wherein, in addition to the safe storage of CO₂, usable products can additionally be obtained. It is also advantageous that NaCl or KCl are obtainable as raw materials simply and inexpensively.

Some embodiments of the invention provide a method for producing chlorine and storing carbon dioxide, wherein
a) a compound of the formula M⁺Cl⁻ is reacted electrolytically to M and Cl₂, wherein M⁺=Na, K or a mixture thereof;
b) M is reacted with carbon dioxide and optionally H₂O to M₂CO₃ and/or optionally to MHCO₃;

and
c) M₂CO₃ and/or optionally MHCO₃ is stored.

Other embodiments of the invention provide a device for producing chlorine and storing carbon dioxide, comprising
an electrolysis device E, which is designed to react a compound of the formula M⁺Cl⁻ electrolytically to M and Cl₂, wherein M⁺=Na, K or a mixture thereof;
a first feed device 1 for M⁺Cl⁻, which is designed to feed M⁺Cl⁻ to the electrolysis device E,
a first discharge device 1′ for Cl₂, which is designed to remove Cl₂ from the electrolysis device E;
a second discharge device 2′ for M, which is designed to remove M from the electrolysis device E;
a first reactor R for reacting carbon dioxide with M, which is designed to react M with carbon dioxide and optionally H₂O to M₂CO₃ and/or optionally to MHCO₃;
a second feed device 2 for M, which is connected to the second discharge device 2′ for M and is designed to feed M to the first reactor R;
a third feed device 3 for carbon dioxide and optionally H₂O, which is designed to feed carbon dioxide and optionally H₂O to the first reactor R;
a third discharge device 3′ for M₂CO₃ and/or optionally MHCO₃, which is designed to remove M₂CO₃ and/or optionally MHCO₃ from the first reactor R; and
a storage device S for storing M₂CO₃ and/or optionally MHCO₃, which is designed to store M₂CO₃ and/or optionally MHCO₃ which comes from the third discharge device 3′.

Some embodiments of the invention are directed to a method for producing chlorine and storing carbon dioxide, wherein
a) a compound of the formula M⁺Cl⁻ is reacted electrolytically to M and Cl₂, wherein M⁺=Na, K or a mixture thereof;
b) M is reacted with carbon dioxide and optionally H₂O to M₂CO₃ and/or optionally to MHCO₃; and
c) M₂CO₃ and/or optionally MHCO₃ is stored.

In principle, the total conversion of this embodiment can be represented in the following equation:

2 MCl+2 CO₂→M₂CO₃+CO+Cl₂, wherein M=Na and K or mixtures thereof.

Carbon monoxide can thus also be produced in the reaction in step b), which is then reacted to give further chemical products.

The CO₂ storage density per metal chloride used doubles if the hydrogen carbonate is sequestered instead of the carbonate.

2 MCl+3 CO₂+H₂O→2 MHCO₃+CO+Cl₂, wherein M=Na and K or mixtures thereof.

In the reaction in step c), the following reactions, for example, are possible, for example if air with a proportion of oxygen, hydrogen and carbon dioxide is used:

2 M+CO₂→M₂O+CO

2 M+2 CO₂→M₂CO₃+CO

2 M+½ O₂→M₂O; M₂O+CO₂→M₂CO₃

2 M+2 H₂O→MOH+H₂ MOH+CO₂→MHCO₃

The CO obtained according to those equations can be removed from the method after step b), that is to say from the reactor for reacting carbon dioxide with M, and serves as a usable substance. For example, it can be reacted with hydrogen in a Fischer-Tropsch process to alcohols or other longer-chained hydrocarbons.

n CO+(2n+1) H₂→CₙH₂ₙ₊₂+n H₂O (alkanes)

n CO+(2n) H₂→CₙH₂ₙ+n H₂O (alkenes)

n CO+(2n) H₂→CₙH₂ₙ₊₁OH+(n−1) H₂O (alcohols)

The reactions proceed exothermally, so that no additional energy has to be used for the process. Thus, carbon monoxide can also be produced in the reaction in step b), which can be reacted to give further chemical products. Those products in turn represent usable substances which can be sold as such or used as starting materials for the chemical industry.

In addition, a reaction of M with nitrogen could optionally also take place in step b), if nitrogen is present in the atmosphere of the reaction. Various products of nitrogen could thereby be formed, which can then react further to ammonia, amines (with CO or alkanes, alkenes, etc.), nitro compounds, etc. Ammonia, for example, can thus also be obtained as a usable substance.

Chlorine gas is additionally formed in the method according to the invention and is removed from step a) and can optionally be stored and/or transported away. According to particular embodiments, it can also be used in situ for further chemical reactions, for example for the chlorination of the alkanes, alkenes, alcohols, etc. produced above, or by reaction with hydrogen to HCl. Cl₂ is used in the chemical industry to produce solvents, intermediates or hydrochloric acid.

The annual production of chlorine is approximately 71 million t (metric tons) in 2013. In the case of the method according to the invention, this corresponds to a sequestrable amount of CO₂ of 88 million t. In addition, there are also 44 million t of CO₂ which leave the process as a whole as CO (28 million t). Based on the chlorine market, the procedure according to the invention thus has a total CO₂ volume of 132 million t/year, which is not released into the atmosphere. At a CO₂ allowance price of 30 [symbol] /t (CO₂), this corresponds to an allowance saving of 3.96 billion [symbol] . At a lower estimated CO₂ emission allowance price of approximately from 7 to 10 [symbol] /t CO₂, the allowance saving alone is nevertheless approximately 1 billion [symbol] . From 28 million t of CO it is possible to produce approximately 14 million t of petroleum, which at a market value of 650 [symbol] /t corresponds to a total volume of a further 9.1 billion [symbol] . The 23 largest German lignite- and hard coal-fired power plants emit approximately 200 million t of CO₂. This means that ⅔ of the waste CO₂ from German power plants could be stored in the ground in the form of a solid if the procedure becomes established and the raw material chlorine is produced by the procedure according to the invention.

According to some embodiments, the compound of the formula M⁺Cl⁻ comes from waste products of the chemical industry and/or is obtained therefrom. For example, such (waste) products can come from salt mining.

There are no particular limitations according to the invention as regards the electrolysis in step a), and it can include, for example, a fused salt electrolysis of a compound of the formula M⁺Cl⁻ or the electrolysis of an aqueous solution of the compound of the formula M⁺Cl⁻.

The use of a fused salt electrolysis of a mixture comprising a compound of the formula M⁺Cl⁻ has the advantage that a higher efficiency is achieved and, in addition, no hydrogen is formed as by-product, which may be undesirable if sufficient hydrogen is present.

On the other hand, when the electrolysis in step a) is carried out by electrolysis of an aqueous solution of the compound of the formula M⁺Cl⁻ with formation of hydrogen, the hydrogen can be used, for example, as a usable product or, according to particular embodiments, can be reacted with carbon monoxide produced in the reaction in step b) to form further chemical products, as illustrated by way of example in the above formulae. Of course, it is also possible that hydrogen for a reaction of the carbon monoxide produced in step b) comes from other sources. The reaction of the carbon monoxide with hydrogen can, for example, also take place in the first reactor, but also in a different reactor.

According to particular embodiments, the reaction of M with carbon dioxide and optionally H₂O in step b) can be carried out by burning M in an atmosphere comprising carbon dioxide and optionally H₂O. However, there are no particular limitations as regards the atmosphere in step b), provided that carbon dioxide is present, and it can also be, for example, air or waste air from a combustion, for example in conventional thermal combustion processes as in coal-fired power plants or in the combustion of mineral oil and/or natural gas. The reaction may take place in an atmosphere which is enriched with carbon dioxide as compared with the normal ambient air, for example waste air from the combustion of carbon-containing materials to produce electrical energy. A combustion in step b) has the advantage of an efficient and rapid reaction process. To that end, the combustion can optionally be started, for example by adding water or by electrical or other ignition sources such as an electric arc, laser, etc.

In particular embodiments, additional thermal energy can be obtained from the reaction in step b), which thermal energy can optionally be used in electrical energy and/or for the preheating of M. The electrical energy can also be used for the electrolysis in step a). According to some embodiments, the energy for the electrolytic reaction of M⁺Cl⁻ to M and Cl₂ is provided substantially from excess energy from renewable energies, that is to say, for example, to the extent of more than 50%, preferably more than 70%, more preferably more than 80% and particularly preferably more than 90%, based on the energy requirement of the electrolysis.

Excess energy from renewable energies is available for that purpose, for example, when more power is made available by renewable and/or conventional energy sources than is purchased by consumers. This means in particular the excess energy which is made available by renewable energy sources such as solar power plants, wind power plants, water power plants, geothermal power plants, biogas power plants (biomass) or the like and which cannot be purchased locally, regionally and/or nationally by consumers at the time of its production. It is possible that energy is also acquired from other sources, for example from conventional power sources and/or from the energy produced above in the reaction in step b). According to some embodiments, 100% of the energy used for the electrolysis of the compound of the formula M⁺Cl⁻ is acquired from renewable energy sources, wherein, for operation of the electrolysis unit, energy that is not directly connected with the electrolysis of the compound of the formula M⁺Cl⁻, such as, for example, for lighting purposes or for operating pumps, etc., can also come from other energy sources, but also from renewable energy sources.

The present invention further includes a device with which the method according to the invention can be carried out.

According to particular embodiments, the invention thus relates to a device for producing chlorine and storing carbon dioxide, comprising

an electrolysis device E, which is designed to react a compound of the formula M⁺Cl⁻ electrolytically to M and Cl₂, wherein M⁺=Na, K or a mixture thereof;

a first feed device 1 for M⁺Cl⁻, which is designed to feed M⁺Cl⁻ to the electrolysis device E,

a first discharge device 1′ for Cl₂, which is designed to remove Cl₂ from the electrolysis device E;

a second discharge device 2′ for M, which is designed to remove M from the electrolysis device E;

a first reactor R for reacting carbon dioxide with M, which is designed to react M with carbon dioxide and optionally H₂O to M₂CO₃ and/or optionally to MHCO₃;

a second feed device 2 for M, which is connected to the second discharge device 2′ for M and is designed to feed M to the first reactor R;

a third feed device 3 for carbon dioxide and optionally H₂O, which is designed to feed carbon dioxide and optionally H₂O to the first reactor R;

a third discharge device 3′ for M₂CO₃ and/or optionally MHCO₃, which is designed to remove M₂CO₃ and/or optionally MHCO₃ from the first reactor R; and

a storage device S for storing M₂CO₃ and/or optionally MHCO₃, which is designed to store M₂CO₃ and/or optionally MHCO₃ which comes from the third discharge device 3′.

According to particular embodiments, the device according to the invention can further comprise a device for generating renewable energy, which is designed to supply electrical energy to the electrolysis device (E). There are no particular limitations as regards the device/plant for generating renewable energy and it can include, for example, wind power plants, water power plants, geothermal power plants, solar power plants, tidal power plants, biothermal power plants or biomass power plants, etc.

In particular embodiments, the first reactor R can have a burner for burning M with carbon dioxide and optionally H₂O. Ignition sources such as light arcs for generating an ignition spark or plasma can also be present for starting the combustion. Further known ignition systems for generating an ignition spark or plasma are, for example, magnetos, electronic igniters and laser igniters.

According to particular embodiments, the first reactor R can also comprise further discharge devices for gaseous products such as CO, NH₃, etc. that are formed.

According to particular embodiments, the electrolysis device E can be a fused salt electrolysis device, or the electrolysis device E can be so designed according to further particular embodiments that an aqueous solution of M⁺Cl⁻ is electrolyzed, wherein the device can then also further comprise a fourth discharge device 4′ for hydrogen, which is designed to remove hydrogen from the electrolysis device E.

The device according to the invention can further comprise a fourth feed device 4 for hydrogen, which according to particular embodiments is connected to the fourth discharge device 4′ for hydrogen and is designed to feed hydrogen to the first reactor R. Of course, a fourth feed device 4 for hydrogen that is not connected to the fourth discharge device 4′ for hydrogen can also be present, or such a fourth feed device 4 for hydrogen can also be present in embodiments in which the fourth discharge device 4′ for hydrogen is not necessarily present, for example if the electrolysis device E is designed for carrying out a fused salt electrolysis.

The device according to the invention can additionally also comprise one or more reservoirs for storing chlorine and/or further products such as carbon monoxide or hydrogen and/or also secondary products such as COCl₂ (CO+Cl₂), HCl (Cl₂+H₂), etc., and/or also further reactors for reacting carbon monoxide with hydrogen and optionally reservoirs for storing products of such a reaction of carbon monoxide and hydrogen, for example alkanes, alkenes and/or alcohols. It is advantageous that these products can be produced in a simultaneous sequestration of carbon dioxide. Any carbon dioxide produced in such further reactions can be fed back to the method according to the invention or alternatively discharged into the atmosphere if waste air having a higher concentration of carbon dioxide, for example from the combustion of carbon-containing compounds, is available for the reaction in step b), or if the electrolysis device E and the first reactor R are spatially far apart from one another.

It is possible according to the invention that the electrolysis device E and the first reactor R are situated at locations that are spatially far apart, for example if the electrolysis device E is situated in the vicinity of plants for generating renewable energy and the first reactor R is situated in a different location, where combustion of carbon dioxide with the metal M or similar reactions have already been carried out previously and it is more advantageous to transport the metal M from the electrolysis device E to the first reactor R, for example by ship, train or truck, than to construct a new first reactor R close to the electrolysis device E, which can be associated with considerable costs. It is also possible that the first reactor R and the storage device S are situated at locations that are spatially far apart, for example if there is not sufficient space in the vicinity of the first reactor R to store the products, such as, for example, M₂CO₃ and/or optionally MHCO₃, that are produced, or if those products also find buyers in a different place as starting materials and it may be preferred to store them in situ with the buyers.

According to particular embodiments, however, the electrolysis device E, the first reactor R and/or the storage device S as well as optionally further reservoirs for further products are not situated too far away from one another, in order to avoid as far as possible the production of carbon dioxide by the transportation to further reservoirs of, for example, M, M₂CO₃ and/or MHCO₃ or other substances that are produced. The electrolysis device E may also be situated in the vicinity of plants for generating renewable energies, which frequently produce excess energy which cannot be purchased locally or regionally. Since this can also fluctuate seasonally, however, it is also possible for different electrolysis devices E to supply the first reactor R with M at different times.

According to the invention, there are no particular limitations as regards the various feed devices, the first feed device 1, the second feed device 2, the third feed device 3 and optionally the fourth feed device 4, as well as the various discharge devices, the first discharge device 1′, the second discharge device 2′, the third discharge device 3′ and optionally the fourth discharge device 4′, and they can include (filling) hoppers, pipes, conveyor belts, etc., but also means of transport such as trucks, ships, freight containers on trains, etc., for example in the case of the feeding of M, which can be suitably provided.

It should additionally be noted that the intermediates alkali metal M, alkali hydroxide solution MOH, which can form in the combustion of M with water, alkali hydrogen carbonate or alkali carbonate are themselves valuable and have hitherto optionally been obtained by different methods. Sodium carbonate, for example, is at present obtained from natural sources, and other products, such as sodium hydroxide solution, are obtained by other chemical processes. Since the method according to the invention yields a substantially larger amount of sodium carbonate or sodium hydrogen carbonate and/or sodium hydroxide solution, it may thus replace or at least reduce, for example, the supply of such substances from natural sources or the preparation thereof by other methods.

These process-related advantages arise in particular when an aqueous alkali metal chloride electrolysis, wherein the alkali metal is K or Na, is replaced by a fused salt electrolysis and the substances are reacted in pure or modified form according to the process sequences. In terms of energy, the entire process is driven in particular embodiments especially by an overproduction of electrical energy, which cannot be fed into the grid.

The method according to the invention can in principle be used at sites which now use the electrolysis of NaCl and or KCl, for example in the case of an aqueous sodium chloride solution, to obtain the raw material chlorine. Hitherto, there has often been no use for the hydrogen formed thereby, wherein it can be used, as shown above, to produce products of higher value such as alkanes, alkenes or alcohols. If too much hydrogen is present, the use of a fused salt electrolysis is advantageous so that, depending on the presence of and demand for hydrogen, an electrolysis of an aqueous solution of M⁺Cl⁻ or a fused salt electrolysis can be carried out alternately, for example, in order to have available hydrogen for the production of products of higher value, for example by the Fischer-Tropsch process, according to the demand and/or requirement.

The method as a whole can accordingly proceed as follows, for example, with any preparative steps that are present:

0) sodium chloride and potassium chloride are often naturally found together and are separated in a relatively complex procedure by electrostatic methods. However, the separation has only limited effectiveness, so that large stocks of potassium/sodium chloride form which can no longer be worked up further for economic reasons.

Electrostatic Method

If, for example, a substance such as the salts NaCl or KCl is rubbed against another material, they can both become “electrically” charged. This principle is used to separate solids mixtures. To that end, the crude salt is ground, for example, to a grain size of one millimeter. In the next step, the salts can be treated with surface-active substances, so that they become selectively positively and negatively charged against one another. The salt crystals then trickle through a “free-fall separator”. This consists of two electrodes, between which there is a high-voltage electric field. The differently charged salts are deflected to the anode or to the cathode. The sorted minerals are collected separately beneath the free-fall separator.

These stocks can then be used as a chloride source, wherein, however, the mixture of KCl and NaCl as such can also be used as the chloride source in step a). Less preferred for this purpose is direct decomposition. If the CO₂ allowances increase in value, direct decomposition may also become economical. The mixtures in question are mixtures comprising different proportions of potassium chloride and sodium chloride, which mixtures tend to have no value. According to the NaCl—KCl phase diagram, the melting points of the salts are lowered considerably in mixtures, which allows the electrolysis to become more advantageous in terms of energy, in particular in the case of a fused salt electrolysis. Typical efficiencies here are, for example, greater than 50%. It is not important for the method whether the alkali metals are separated off in pure form or in the form of an alloy of the individual components. Further salt additions (for example CaCl₂) to lower the melting point are likewise possible.

1) In the second step, the alkali metals are produced from the halides by, for example, fused salt electrolysis, as is already known.

A sodium electrolysis according to the prior art can take place, for example, in the production of sodium by fused salt electrolysis of dry sodium chloride in a so-called Downs cell. In order to achieve a more efficient electrolysis at lower temperatures, the melting point can be lowered, for example, by using a eutectic salt mixture of 60% calcium chloride and 40% sodium chloride, which melts at 580° C. Barium chloride is also possible as an addition. After a suitable voltage, for example approximately seven volts, has been applied, the following processes, for example, take place:

Cathode:

2Na⁺+2e⁻→2Na⁰

Anode:

2Cl⁻→Cl₂+2e⁻

Overall Reaction:

2Na⁺+2Cl⁻→2Na+Cl₂

For example, for the production of one kilogram of sodium, approximately 10 kWh of current are consumed during the electrolysis according to a method of the prior art.

Analogous reactions are found for KCl and/or mixtures of NaCl and KCl.

2) The reaction in step b) then takes place, for example, according to the following equations:

2 M+CO₂→M₂O+CO

2 M+2 CO₂→M₂CO₃+CO

2 M+½ O₂→M₂O; M₂O+CO₂→M₂CO₃

2 M+2 H₂O→MOH+H₂ MOH+CO₂→MHCO₃

In the combustion of the alkali metals in atmospheres comprising, for example, CO₂, N₂, O₂, H₂O, there can thus be obtained heat which can be used in the device or in the power plant comprising the first reactor R, for example also for preheating M, and valuable synthesis building blocks such as CO, H₂.

In order to increase the storage density, the metal carbonate can be reacted with water and further CO₂ to give metal hydrogen carbonate:

M₂CO₃+H₂O+CO₂→2 MHCO₃

The metal carbonates or metal hydrogen carbonates can subsequently be used or, in the case of the expected overproduction, sequestered.

The above embodiments, forms and further developments can, where expedient, be combined with one another as desired. Further possible forms, further developments and implementations of the invention also include combinations, not mentioned explicitly, of features of the invention that are described hereinabove or herein below in relation to the exemplary embodiments. In particular, the person skilled in the art will also add individual aspects as improvements or additions to the particular basic form of the present invention.

### EXAMPLES

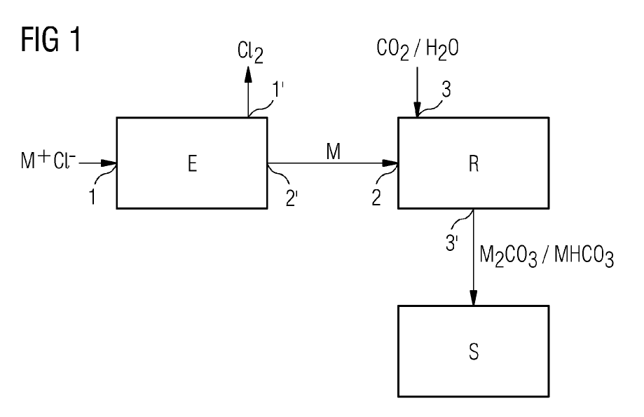

Exemplary embodiments of a plant are shown in FIGS. 1 to 3.

### Example 1

A first embodiment of a device according to the present invention is shown in FIG. 1.

A compound M⁺Cl⁻ is fed via a first feed device 1 for M⁺Cl⁻ to the electrolysis device E. This is in the form of fused salt electrolysis, for example. In the electrolysis device, the compound is then electrolyzed to Cl₂ and M, wherein the Cl₂ is removed from the electrolysis device E via a first discharge device 1′ for Cl₂ and can then be stored, transported away or used further. M is additionally removed from the electrolysis device E via a second discharge device 2′ for M. This is connected to a second feed device 2 for M, by means of which M is introduced into the first reactor R for the reaction of carbon dioxide. Furthermore, carbon dioxide and optionally H₂O are fed to the first reactor R via the third feed device 3 for carbon dioxide and optionally H₂O. In the first reactor, M is reacted with CO₂ and optionally H₂O as well as optionally further gases such as N₂ or O₂, which are not shown, to give M₂CO₃ and/or optionally MHCO₃ as well as optionally further products, such as MOH, NH₃, etc., which are not shown. The M₂CO₃ and/or optionally MHCO₃ produced are removed from the first reactor R via a third discharge device 3′ for M₂CO₃ and/or optionally MHCO₃ and introduced into a storage device S for storing M₂CO₃ and/or optionally MHCO₃. M₂CO₃ and/or optionally MHCO₃ can optionally be removed from the storage device S if there is a corresponding demand. The gaseous products produced in the first reactor R can also be removed from the first reactor R via a discharge device (not shown).

### Example 2

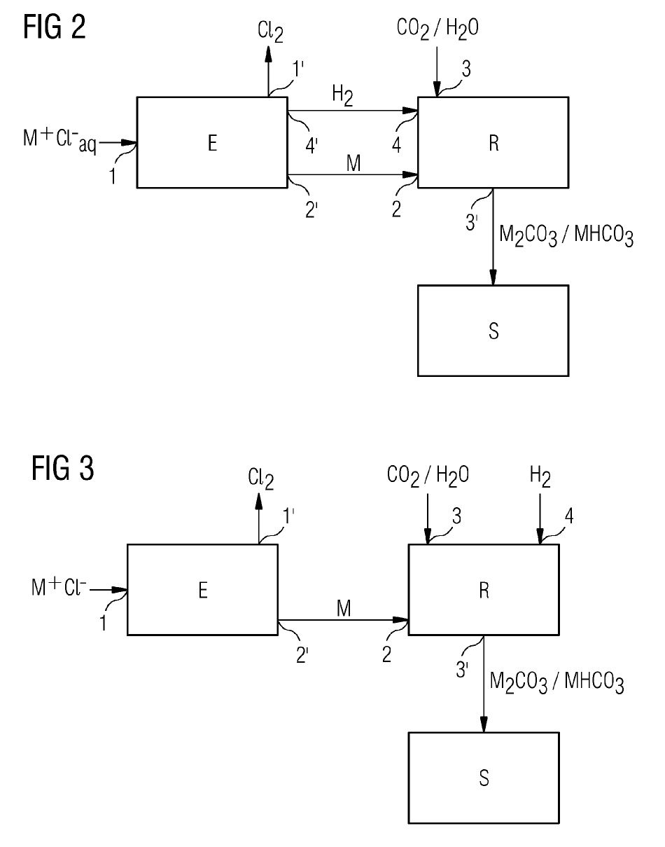

The device of Example 2 is shown in FIG. 2 and corresponds to that of Example 1, wherein the fused salt electrolysis as the electrolysis device E is replaced by one in which an aqueous solution of M⁺Cl⁻ is electrolyzed. Hydrogen thereby forms in the electrolysis device E and is removed from the electrolysis device E via a fourth discharge device 4′ for hydrogen. The fourth discharge device 4′ for hydrogen is connected to a fourth feed device 4 for hydrogen, by means of which the hydrogen is fed to the first reactor R, wherein alkanes, alkenes, alcohols, etc. can then be produced with the CO. These can also be removed via the discharge device (not shown) for gaseous products or via yet a further discharge device.

### Example 3

The device of Example 3 shown in FIG. 3 corresponds to the device in Example 1, wherein hydrogen is fed to the first reactor R via a fourth feed device 4 for hydrogen. Hydrogen can again be reacted with CO to give alkanes, alkenes, alcohols, etc., which can be removed via the discharge device (not shown) for gaseous products or via yet a further discharge device.

According to the present invention there is described an overall concept which
a. uses as the material source substances which occur naturally as chlorides such as sodium chloride or potassium chloride;
b. preferably by means of excess electrical energy receives the alkali metals sodium and potassium as material seasonal energy stores by electrolysis and chlorine for chlorine production;
c. burns those metals with CO₂ to give sodium carbonate or potassium carbonate, with the formation of CO;
d. sequesters sodium carbonate or potassium carbonate or hydrogen carbonate, whereby CO₂ is removed from the atmosphere. The amount that can be sequestered is governed by the excess energy present and the chlorine requirement;
e. supplies the chemical industry with the basic chemical chlorine for the production of solvents, intermediates or hydrochloric acid.

Coupling chlorine production to the seasonal material energy stores of alkali metals opens up the possibility of a process for sequestering CO₂ in the form of metal carbonate or metal hydrogen carbonate.

This process additionally allows carbon capture and storage to be coupled to the time- and location-based overproduction of renewable energies. Excess energy is stored in the form of alkali metals. Discharging this store in conjunction with CO₂ yields thermal energy at a high temperature level for reconversion and additionally valuable intermediates for chemical synthesis.

At the end of the use chain, solid, non-toxic metal carbonate and/or metal hydrogen carbonate suitable for landfill is formed. The maximum possible volume is probably limited to the worldwide chlorine requirement but would be sufficient to permanently store ⅔ of the CO₂ emitted by the 24 largest German lignite- and coal-fired power plants.

Additional financial benefit is obtained by the saving made in terms of CO₂ allowances and the production of valuable products such as petroleum or alcohols.

What is claimed is:

1. A method for producing chlorine and storing carbon dioxide, the method comprising:
a) electrolytically reacting a compound of the formula M⁺Cl⁻ to produce M and Cl₂, wherein M⁺=Na, K, or a mixture of Na and K;
b) reacting the produced M with at least carbon dioxide to produce at least M₂CO₃; and
c) storing the produced M₂CO₃.

2. The method of claim 1, wherein the reaction of M with at least carbon dioxide in step (b) includes burning M in an atmosphere comprising carbon dioxide.

3. The method of claim 1, wherein the electrolytic reaction of M⁺Cl⁻ to M and Cl₂ in step (a) is performed using excess energy from at least one renewable energy.

4. The method of claim 1, wherein the compound of the formula M⁺Cl⁻ is obtained from chemical industry waste products.

5. The method of claim 1, wherein the electrolysis in step (a) is includes fused salt electrolysis of a mixture comprising a compound of the formula M⁺Cl⁻.

6. The method of claim 5, wherein the reaction at step (b) also forms carbon monoxide, which is reacted to give further chemical products.

7. The method of claim 1, wherein the electrolysis in step (a) includes electrolysis of an aqueous solution of the compound of the formula M⁺Cl⁻ with the formation of hydrogen.

8. The method of claim 7, wherein the reaction at step (b) also produces carbon monoxide, which is reacted with the hydrogen formed at step (a) to form further chemical products.

9. A device for producing chlorine and storing carbon dioxide, the device comprising:
an electrolysis device configured to electrolytically react a compound of the formula M⁺Cl⁻ to produce M and Cl₂, wherein M⁺=Na, K or a mixture thereof;
a first feed device configured to feed M⁺Cl⁻ to the electrolysis device,
a first discharge device configured to remove Cl₂ from the electrolysis device;
a second discharge device configured to remove M from the electrolysis device;
a first reactor configured to react M with at least carbon dioxide to produce at least M₂CO₃;
a second feed device connected to the second discharge device and configured to feed M to the first reactor;
a third feed device configured to feed at least the carbon dioxide to the first reactor;
a third discharge device configured to remove M₂CO₃; and
a storage device configured to store at least the M₂CO₃ removed by the third discharge device.

10. The device of claim 9, further comprising a device configured to generate renewable energy, and to supply electrical energy to the electrolysis device.

11. The device of claim 9, wherein the first reactor has a burner for burning M with the carbon dioxide.

12. The device of claim 9, wherein the electrolysis device is a fused salt electrolysis device.

13. The device of claim 9, wherein the electrolysis device is configured to electrolyze an aqueous solution of M⁺Cl⁻, and the device further comprises a fourth discharge device configured to remove hydrogen from the electrolysis device.

14. The device of claim 13, further comprising a fourth feed device connected to the fourth discharge device and configured to feed hydrogen to the first reactor.

15. The method of claim 1, wherein step (b) includes reacting M with carbon dioxide and H₂O to produce M₂CO₃.

16. The method of claim 1, wherein step (b) includes reacting M with carbon dioxide and H₂O to produce M₂CO₃ and MHCO₃.

17. The method of claim 1, wherein step (b) includes reacting M with at least carbon dioxide to produce M₂CO₃ and MHCO₃.
